(19) United States
(12) Patent Application Publication
(10) Pub. No.: US 2021/0080298 A1
(43) Pub. Date: Mar. 18, 2021

(54) APPARTUS FOR AUTOMATICALLY MEASURING THE FLOW OF POWDERS AND GRANULAR MATERIALS THROUGH ORIFICES UNDER DYNAMIC CONDITIONS

(71) Applicant: Gregory Peter Martiska, Newtown, CT (US)

(72) Inventor: Gregory Peter Martiska, Newtown, CT (US)

(21) Appl. No.: 16/569,883

(22) Filed: Sep. 13, 2019

(51) Int. Cl. G01F 1/00; G01F 1/34

(52) U.S. Cl. CPC G01F 1/007; G01N 2011/008; G01F 1/34

(57) ABSTRACT

An apparatus is provided for measuring the flow of powders and granular materials through orifices under dynamic conditions. The apparatus consists of a container for receiving a material sample to be investigated with one or more orifices, a means of moving the container to initiate flow in the material sample and to allow it to flow through the orifices in the container, and a means of measuring the amount of material flowing through the orifices. The amount and rate of material flowing through the orifices is a measure of the flowability of the material sample.

### CROSS-REFERENCE TO RELATED APPLICATION

Not Applicable

### BACKGROUND OF THE INVENTION

### 1) Field of the Invention

This invention relates to an apparatus for automatically measuring the flow of powder and granular materials through orifices under dynamic conditions. The information produced by these measurements can determine how well a material performs in various processes and equipment such die filling machines, powder injection molds, and pharmaceutical tableting machines.

### 2) Description of the Related Art

Many apparatus have been disclosed and produced for measuring how powders and granular materials flow through orifices. These devices range from funnels with a fixed size opening to sample cups with various size orifices in the bottom. The problem with these devices is that the material sample is typically in a static, non-moving condition at the start of the test. This does not simulate many real world applications where the sample powder is moving when it must flow through an opening in a processing machine. This is sometimes solved by manually pouring the material sample into the device at the beginning of the test but this is difficult to control. Also some of these devices use a motorized blade to stir the sample powder to create flow but this does not well simulate powder flowing under gravitational force. Additionally these devices are typically manual devices and results can vary depending on the operator.

A typical funnel device is called the hall funnel and is described in ASTM Standard B213-17. This device consists of a funnel with an opening of 1 millimeter. The opening is blocked by the finger of the operator and sample funnel is filled with a known amount of material sample. The operator removes their finger and measure the time it takes for the sample to flow from the funnel. The device can also be automated for measuring the flow time using a detector for the sample. The problem with this technique is that the powder is not moving at the beginning of the test. This means the test does not simulate how a sample moves through the opening when it is already moving. Also the measurement recorded is time in seconds and not the amount of material that has flowed through the funnel.

Another funnel device is called the Carnet funnel and is described in ASTM B964-16. This device consists of a funnel with an opening of 5 millimeters. The opening is blocked by the finger of the operator and sample funnel is filled with a known amount of material sample. The operator removes their finger and measure the time it takes for the sample to flow from the funnel. The device can also be automated for measuring the flow time using a detector for the sample. The problem with this technique is that the powder is not moving at the beginning of the test. This means the test does not simulate how a sample moves through the opening when it is already moving. The sample can be poured into the funnel manually to attempt to simulate dynamic sample flow conditions but this is difficult to control and the powder is only moving as it is poured into the funnel. Also the measurement recorded is time in seconds and not the amount of material that has flowed through the funnel.

Another device for testing powder flow properties is described in the paper Intrinsic flowability: A new technology for powder-flowability classification by Alberto Gioia in Pharmaceutical Technology, February 1980. This device consists of a sample cylinder to which a disc with a hole is attached. The hole is blocked and the cylinder is filled with a material sample. The hole is then unblocked and the sample material is allowed to flow through the hole. The test is repeated with discs with smaller and smaller holes until the powder stops flowing and the maximum hole size that allows flow is recorded. The problem with this test is that is does not simulate dynamic powder flow conditions but only static conditions where the powder transitions from not moving to moving.

### BRIEF SUMMARY OF THE INVENTION

After designing several powder flow testers including apparatus described in U.S. Pat. Nos. 8,438,914 and 8,335,343, the inventors of the present invention determined that industry and academia needed an apparatus to measure how powders and granular materials flow through orifices under dynamic flow conditions and under a range of flow speeds. This would allow researchers to simulate powder flow behavior in many industrial material handling machines.

The invention consists of a container for receiving a material sample to be investigated with one or more orifices, a means of moving the container so the material sample moves and can flow through the orifices in the container, and a means of measuring the amount of material flowing through the orifices. The amount and rate of material flowing through the orifices is a measure of the flowability of the material sample.

### BRIEF DESCRIPTION OF THE DRAWINGS

FIG. 1 presents a drawing of the preferred embodiment of the sample container.

FIG. 2 presents a drawing of the preferred embodiment of the apparatus for moving the container and sensing the powder flowing through the opening.

FIG. 3 presents a drawing of the preferred embodiment of the apparatus with the sample container lid in the measurement position.

### DETAILED DESCRIPTION OF THE INVENTION

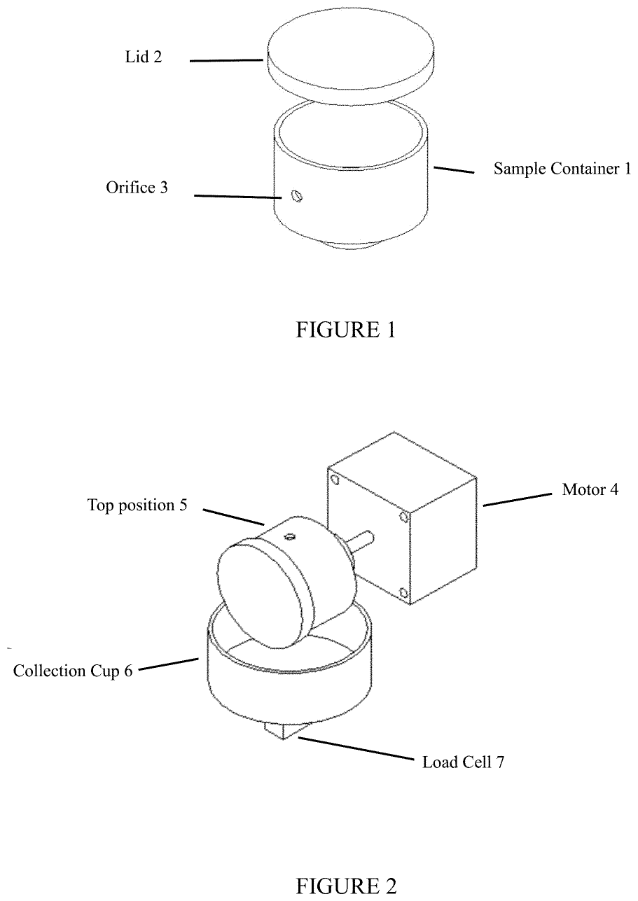

The preferred embodiment of the invention is shown in FIG. 1 and FIG. 2. The apparatus consists of a cylindrical sample container 1 with an attachable lid 2 and an orifice or hole in the side wall 3. A material sample of powder or granular material is poured into the sample container in a manner so that it does not exit through the orifice in the side. The lid is then attached to the sample container and the sample container is mounted horizontally on a motor 4 shaft with the orifice at the top position 5. The motor 4 is then started and the sample container is rotated along its axis so then material sample flows and the orifice in the side of the container passes under the material sample over and over again. Any material passing through the orifice is collected in a collection cup 6 that is resting on a load cell 7 that can measure the mass of material in the collection cup. The mass is recorded over time as the sample container is rotated until powder stops flowing from the sample container or for a fixed time. The test can be repeated at various continuous rotation rates or the sample container can be rotated until the hole is under the center of the material sample and the rotation is then stopped.

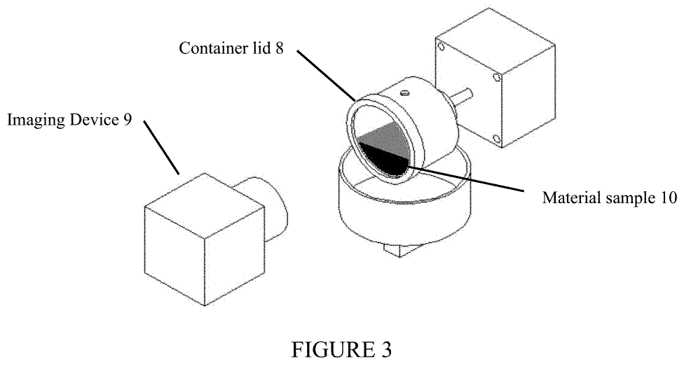

FIG. 3 shows an additional embodiment of the invention that uses an imaging device to measure the amount of material flowing through the orifice in the sample container. The container lid 9 and container bottom are transparent to the imaging device so it can sense the material sample 10 inside the container. The sample container is then rotated and images of the material sample are taken at various intervals. Using image analysis software, the volume of sample in the container at any given time can be determined from the cross-sectional area of the material sample and the length of the container. The volume of the sample is recorded over time as the sample container is rotated until the material sample stops flowing from the sample container or for a fixed time. The volume drops as material sample flows through the orifice and out of the sample container.

Additional embodiments would consist of a sample container that is not cylindrical but rectangular, pyramidal, or funnel shaped.

What is claimed is:

1. An apparatus for testing the flow of powder and granular material through orifices comprising:
a) a sample container for receiving a material sample to be investigated with one or more orifices through which the material sample can flow;
b) a means of moving the sample container so the material sample flows and can flow through the orifice or orifices in the container;
c) a measuring unit with a means of measuring the amount of material flowing through the orifice or orifices in the container over time.

2. An apparatus according to claim 1 where said measuring unit consists of a sensor and a measuring circuit that measures the mass of the material sample flowing through the orifice or orifices in the sample container.

3. An apparatus according to claim 1 where said measuring unit consists of an imaging device that can take images of the material sample and an analysis unit that can calculate the volume of the material sample in the container over time from the images and thus determine the volume of material sample flowing through the orifice or orifices in the sample container.

4. An apparatus according to claim 1 where said measuring unit consists of a sensor and measuring circuit that measures the mass of material sample flowing through the orifice or orifices in the sample container and an imaging device that can take images of the material sample and an analysis unit that can calculate the volume of the material sample in the container over time from the images and thus determine the volume of material sample flowing through the orifice or orifices in the sample container.
